(19) United States
(12) Patent Application Publication
(10) Pub. No.: US 2026/0286339 A1
(43) Pub. Date: Sep. 24, 2026

(54) PREPARATION AND COMPOSITIONS OF COT-1 NUCLEIC ACID

(71) Applicant: ILLUMINA, INC., San Diego, CA (US)

(72) Inventors: Jian-Sen Li, San Diego, CA (US); Xin Sheng, San Diego, CA (US); Angelica Marie Barr Schalembier, San Diego, CA (US); Stephen Gross, San Diego, CA (US)

(21) Appl. No.: 19/423,675

(22) Filed: Dec. 17, 2025

Related U.S. Application Data
(63) Continuation of application No. PCT/US2024/059144, filed on Dec. 9, 2024.
(60) Provisional application No. 63/607,488, filed on Dec. 7, 2023.

(30) Foreign Application Priority Data
Dec. 9, 2024 (WO) PCT/US2024/059144

(51) Int. Cl. C12N 15/10; C12Q 1/6806

(52) U.S. Cl. CPC C12N 15/1013; C12N 15/1065; C12Q 1/6806

(57) ABSTRACT

Presented herein are Cot-1 methods and compositions that may be used for reducing non-specific binding, such as enhancing specific enrichment of target sequences in a nucleic acid library. In an embodiment, a Cot-1 composition may be a modified Cot-1 composition that has sequences with similarity to coding sequences removed. In an embodiment, a Cot-1 composition may be a synthetic Cot-1 composition. In an embodiment, a Cot-1 composition may be a minimal or reduced Cot-1 composition.

### CROSS-REFERENCE TO RELATED APPLICATIONS

The present application is a bypass continuation of PCT/US2024/059144, filed Dec. 7, 2023, claims priority to and the benefit of U.S. Provisional Application No. 63/607,488, filed on Dec. 7, 2024, which are incorporated by reference herein in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 4, 2025, is named “ILUM0134PCT.xml” and is 1,850,152 bytes in size. The sequence listing contained in this .XML file is part of the specification and is hereby incorporated by reference herein in its entirety.

### BACKGROUND

The present disclosure relates generally to Cot-1 hybridization blockers, such as those for reducing off-target capture of nucleic acids in various assays.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Certain analysis techniques rely on hybridization of nucleic acids to their complements. For example, targeted sequencing methods use a panel or set of probes that hybridize to target sequences in the nucleic acid library, which may also contain off-target sequences that are not of interest. Hybridization of the probes to the target sequences allows these sequences to be separated from the rest of the fragments in the library for sequencing. By targeting only a portion of the nucleic acid library, hybrid capture methods avoid sequencing of the off-target nucleic acid fragments that do not contain sequences of interest. Off-target reads not only waste sequencing yield, but also potentially compromise variant calling for somatic mutations of low frequency. However, certain probes may be associated with non-specific binding. In such cases, hybridization blockers, such as Cot-1 DNA, may be used to reduce non-specific binding.

### BRIEF SUMMARY

Provided herein is a method of generating Cot-1 DNA that includes contacting genomic DNA fragments with a hybridization probe panel under conditions that permit hybridization of probes of the hybridization probe panel to the genomic DNA fragments, wherein the probes of hybridization probe panel are configured to hybridize to coding regions of the genomic DNA fragments; separating a first portion of the genomic DNA fragments from a second portion of the genomic DNA fragments based on differential binding to the hybridization probe panel, wherein the first portion is not bound to the probes and the second portion is bound to the probes; and providing the first portion of the genomic DNA fragments as Cot-1 DNA.

Provided herein is also a method of generating Cot-1 DNA that includes providing genomic DNA fragments; exposing the genomic DNA fragments to a denaturing temperature to yield denatured DNA; allowing the denatured DNA to reanneal to yield reannealed DNA; and purifying the reannealed DNA using solid-phase reversible immobilization beads; and providing the purified DNA as Cot-1 DNA.

Provided herein is also a method of providing a minimal Cot-1 composition that includes providing probe sequence information of a panel of probes configured to hybridize to targeted regions of genomic DNA; identifying, in the probe sequence information, sequences that overlap with repetitive element sequences in the genomic DNA; and providing a plurality of oligonucleotides having a length of 60-100 nucleotides that correspond to the repetitive elements sequences as the minimal Cot-1 composition.

Provided herein is also a Cot DNA composition prepared by a method comprising: contacting genomic DNA fragments with a hybridization probe panel under conditions that permit hybridization of probes of the hybridization probe panel to the genomic DNA fragments, wherein the probes of hybridization probe panel are configured to hybridize to coding regions of the genomic DNA fragments; separating a first portion of the genomic DNA fragments from a second portion of the genomic DNA fragments based on differential binding to the hybridization probe panel, wherein the first portion is not bound to the probes and the second portion is bound to the probes; and providing the first portion of the genomic DNA fragments as the Cot DNA composition.

Provided herein is also a synthetic Cot-1 DNA composition comprising any combination of SEQ ID NOS: 1-1364.

Provided herein is also a synthetic Cot-1 DNA composition comprising a plurality of oligonucleotides having a length of 60-100 nucleotides and comprising SEQ ID NOS: 1-1364.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 is a schematic illustration of an example target enrichment sequencing technique with or without a Cot-1 hybridization blocker, according to an embodiment of the present disclosure;

FIG. 2 is a flow diagram of a Cot-1 preparation technique, according to an embodiment of the present disclosure;

FIG. 3 is a schematic illustration of a Cot-1 preparation technique, according to an embodiment of the present disclosure;

FIG. 4 is a flow diagram of a Cot-1 preparation technique, according to an embodiment of the present disclosure;

FIG. 5 is a schematic illustration of a Cot-1 preparation technique, according to an embodiment of the present disclosure;

FIG. 6 is a flow diagram of a Cot-1 preparation technique, according to an embodiment of the present disclosure;

FIG. 7 is a schematic illustration of a Cot-1 preparation technique, according to an embodiment of the present disclosure e;

FIG. 8 shows padded read enrichment data for a minimal Cot-1 composition as discuss herein; and

FIG. 9 is a block diagram of a sequencing device configured to acquire sequencing data, according to an embodiment.

### DETAILED DESCRIPTION

Hybridization probes may have imperfect specificity for their nucleic acid targets. For example, in an exome sequencing reaction, certain hybridization probes may pull down intronic or intergenic sequences from a nucleic acid library along with target sequences. These off-target fragments, once pulled down, are then present in the pool of nucleic acid fragments that are sequenced, which results in incorrect sequences being present in the sequence data. The use of hybridization blockers, such as Cot-1 DNA, may block certain types of off-target binding.

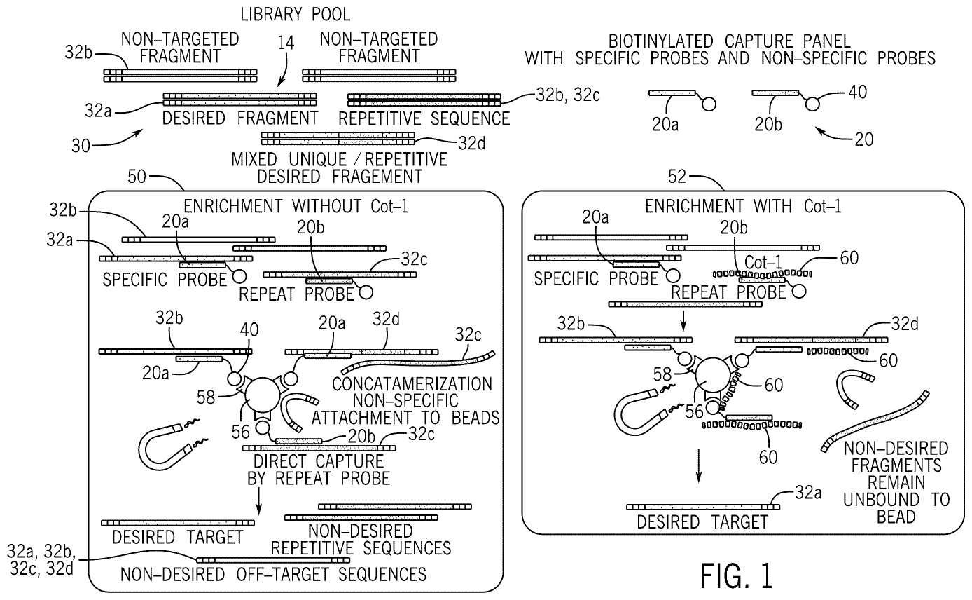

FIG. 1 is a schematic representation of a target enrichment capture workflow with or without Cot-1 to reduce off-target binding. The workflow includes preparation of a nucleic acid library 30 formed from a plurality of nucleic acid fragments 32 from the sample, such as a sample including genomic DNA (e.g., a human genome, an animal genome, a bacterial genome) or other nucleic acids. The nucleic library includes fragments having sequences from regions of interest (e.g., fragment 32a) that include target sequences (e.g., target sequence 14) as well as fragments that are off-target (e.g., fragment 32b) having only off-target sequences. Among the off-target fragments 32b may be certain repetitive sequence fragments 32c that may be subject to off-target binding. It should be understood that fragments 32a that include target sequences may be formed entirely from regions of interest or may include other regions that are not of interest such that the library 30 may include mixed target fragments 32d.

The target-specific hybridization probes 20 are designed to be complementary to one or more target sequences 14 on the fragments 32s. Accordingly, under hybridization conditions, one or more target-specific hybridization probes 20 will bind to the complementary target sequences 14. This facilitates separation, using probe-linked affinity tags 40, of fragments 32a that have the target sequences 14 from the fragments 32b that do not have the target sequences 14 to create a target-enriched sample for sequencing. However, among the probes 20, there may be a subset of probes 20a with high specificity for target sequences 14 and that do not bind to off-target sequences in the fragments 32b. Another subset of the probes 20b may be a non-specific subset that has some non-specific binding to sequences present in the repetitive sequence fragments 32c and/or the mixed fragments 32d.

Accordingly, as shown in panel 50, when the probes 20 are contacted with strands of the fragments 32, the specific subset 20a binds to on-target fragments 32a with the non-specific subset has at least some binding to the repetitive sequence fragments 32c. Thus, at a separation step (e.g., using beads 56 with affinity tag binders 58), not only are the target fragments 32a captured, but there is also some capture of off-target repetitive sequence fragments 32c. In addition, in some cases, the beads 56 may have some non-specific binding ability to off-target fragments 32b. Accordingly, after separation, the captured strands include strands from on-target fragments 32a, which may include mixed fragments 32d as well as from off-target fragments 32b, including repetitive sequence fragments 32c. These undesired strands from off-target fragments 32b can then enter the subsequent sequencing steps of the workflow to generate undesired sequence data.

In contrast, when a Cot-1 hybridization blocker is provided in the workflow, as shown in panel 52, the Cot-1 DNA interacts with the non-specific subset of probes 20b to reduce off-target fragment capture. In addition, the Cot-1 DNA may interact directly with the beads 56 to reduce another source of non-specific binding. Thus, in the illustrated example, the beads 56 provide captured strands from on-target fragments 32a with a reduction in a presence of off-target fragment strands from off-target fragments 32b.

Cot-1 is a useful hybridization blocker that reduces non-specific binding in target enrichment and other probe-based workflows, such as comparative genomic hybridization (CGH), microarray assays, hybridization pulldowns, in situ hybridization (e.g., FISH), Cot-1 is prepared by denaturing and renaturing large amounts of genomic DNA fragments, followed by purification of the initial duplex DNA fraction. Cot-1 is thus the fraction of the genome that is generally rich in repetitive elements and the like. Cot-1 DNA can be prepared from a eukaryotic (e.g., mammalian, human) genomic DNA source, such as placental DNA, by shearing, denaturing, and reannealing under conditions that enrich repetitive elements. The Cot-1 fraction of human genomic DNA consists largely of rapidly annealing repetitive elements. Repetitive genomic DNA sequences can make up a large fraction of a source genome. For example, more than 40% of the human genome is estimated to be these repetitive sequences. While the fraction may vary in other eukaryotes, many genomes also contain large fractions of these repetitive sequences.

Repetitive sequences may be characterized by their reannealing kinetics after denaturation, which are typically faster than those of low copy sequences. This may be a result of the lower complexity of the repetitive sequences. Cot or C0t analysis is a technique that measures how much repetitive DNA is in a DNA sample such as a genome. Cot analysis involves heating a sample of genomic DNA to a denaturation temperature following by a temperature decrease to permit reannealing of the denatured strands and monitoring reannealing of the DNA. A sequence of single-stranded repetitive sequence DNA is more likely to find a complementary strand because of their relative abundance compared to lower copy sequences, such that common sequences renature more rapidly than rare sequences. The rate of reannealing is proportional to the number of copies of that sequence in the DNA sample, and a sample with a high fraction of repetitive sequences will renature rapidly, while complex sequences will renature more slowly relative to one another. The amount of renaturation or reannealing is measured relative to a Cot or C0t value.

For a concentration C of single-stranded (e.g., denatured) DNA at time t (e.g., time in seconds Ts), the loss of single-stranded DNA may be expressed as −dC/dt=kC², where k is the rate constant in in liters (mole nt)-1 sec-1. A Cot-1 value may be, in embodiments, expressed as Cot-1=1=mol/L×Ts. Thus, a particular sample of DNA (or nucleic acids) may have a Cot-1 value based on the initial concentration of DNA (calculated in moles of nucleotide per liter and assuming an average molecular weight of 339 g/mol). In one example starting at a DNA concentration of 300 ng/L, for 1 mole of dNTP equal to an average of 339 g/mol, then moles of sheared DNA is equal to (0.300 g/L)/(339 g/L)=8.85×10⁻⁴ mol/L. Thus, for this example, the reannealing reaction must proceed for 1130 s in order for Cot to equal 1. However, the reannealing time may vary depending on the initial nucleic acid concentration and, in some cases, the GC composition of the source genomic DNA. The reannealing reaction can be stopped by alcohol precipitation. While certain embodiments of the disclosure relate to Cot-1 DNA having a COT value of 1, it should be understood that other Cot values may also be encompassed in the disclosed techniques (e.g., Cot-1.2, Cot-1.5, Cot-2). Further, Cot DNA may refer to a reannealed DNA composition as discussed herein with a Cot value associated with at least 50%, at least 80%, at least 95%, or at least 99% reannealing of the source DNA. In certain embodiments, the Cot DNA may be prepared from a genomic DNA source and/or may be synthetic. In an embodiment, the Cot DNA source is human placental DNA or mammalian placental DNA, such as male placental DNA.

Cot-1 prepared from genomic DNA may have variable quality and effectiveness as a hybridization blocker due to variability of the source genomic DNA, the reannealed fragment size, and/or the preparation workflow. Provided herein are Cot DNA (e.g., Cot-1 DNA) composition and preparation techniques that provide higher quality and/or more consistent Cot DNA. Cot DNA or Cot-1 DNA may refer to a heterogenous DNA composition. In an embodiment, Cot DNA is double-stranded or mostly double-stranded. The DNA composition may include mostly repetitive elements as generally discussed herein. Cot DNA may, in embodiments, refer to a particular preparation technique and the composition yielded from the preparation technique. Cot DNA may be non-naturally occurring. In one example, a Cot composition that includes DNA fragments within a particular size band (e.g., 100 bp-1000 bp) and that is enriched for repetitive elements does not occur in nature and is generally from modification and/or manipulation of a genomic DNA source.

In one approach, Cot DNA may be improved by removing low copy sequences, such as coding or exome sequences, such that a reannealed fraction is artificially enriched for repetitive sequences. As discussed herein, repetitive elements in DNA fragments tend to reanneal more quickly, because they are more likely to find complements due to their relative abundance in the genome. Thus, by selecting appropriate annealing and reaction times, Cot DNA may be enriched for repetitive elements, while low copy sequences are less likely to be part of the reannealed fragment population. Nonetheless, some coding regions may reanneal to one another and form part of heterogeneous Cot DNA compositions. When Cot DNA is provided as a hybridization blocker, the Cot DNA is formed from an exogenous source. Therefore, it is not desirable to have coding regions in the Cot DNA, because these regions may bind to or associate with hybridization probes in a variety of assay contexts, decreasing the available probes to bind to target DNA or RNA of interest. Because hybridization blockers may be provided in excess concentrations relative to target nucleic acids, the depletion of probes may impact the signal able to be generated from coding regions of the target DNA or RNA, particularly for low concentration sequences. This is turn may impact the accuracy of sequencing results.

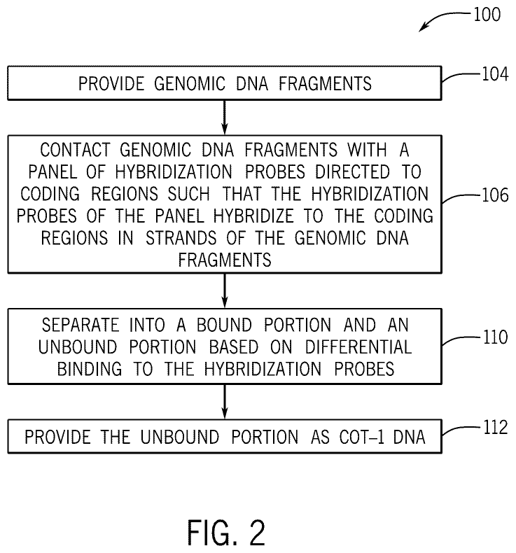

FIG. 2 is a flow diagram 100 of a Cot-1 preparation technique that includes an enrichment step to remove low copy sequences from reannealed genomic DNA fragments, such as coding sequences, during preparation. At step 104, genomic fragments, e.g., genomic DNA fragments, are provided. The fragmentation may be via shearing, sonication, acoustic shearing, enzymatic fragmentation, chemical fragmentation, etc. The fragments may be of a suitable size, such as 50 bp to 1000 bp. In an embodiment, the fragments may undergo a size selection step to exclude fragments outside of a desired size range.

At step 106, the DNA fragments are contacted with a set of probes for a removal step under conditions that permit hybridization. The conditions that permit hybridization may include an initial denaturing step to separate strands of double-stranded fragments (e.g., temperatures above 75° C., above 80° C., in a range of 75° C.-98° C.) and contact with the probes at a subsequent hybridization temperature that is lower than the denaturing temperature. In an embodiment, the hybridization temperature is high enough to prevent non-specific binding. In an embodiment, the hybridization temperature is 45-60° C.

A variety of hybridization or washing conditions include variations in temperature or buffer composition and can be varied according to the specificity of the reaction needed. A range of stringency includes, for example, high, moderate or low stringency conditions. In an embodiment, stringent conditions are selected to be about 5-10° C. lower than the thermal melting point (Tₘ) for the specific sequence at a defined ionic strength and pH. The Tₘ is the temperature, under defined ionic strength, pH and nucleic acid concentration, at which 50% of the probes complementary to the target hybridize to the target sequence at equilibrium. Differences in the number of hydrogen bonds as a function of base pairing between perfect matches and mismatches can be exploited as a result of their different Tₘs. Accordingly, a hybrid comprising perfect complementarity will melt at a higher temperature than one comprising at least one mismatch, all other parameters being equal. Stringent hybridization conditions may also include those in which the salt concentration is less than about 1.0 M sodium ion, generally about 0.01 to 1.0 M sodium ion concentration or other salts at pH 7.0 to 8.3 and the temperature is at least about 30° C. for short probes such as 10 to 50 nucleotides and at least about 60° C. for long probes such as greater than 50 nucleotides. Low stringency conditions include NaCl concentrations of about 1.0 M. Furthermore, low stringency conditions can include MgCl₂ concentrations of about 10 mM, moderate stringency of about 1-10 mM, and high stringency conditions include concentrations of about 1 mM. Stringent conditions also can be achieved with the addition of helix destabilizing agents such as formamide. For example, low stringency conditions include formamide concentrations of about 0 to 10%, while high stringency conditions utilize formamide concentrations of about 40%.

In an embodiment, the probes are a panel of probes representative of coding regions in the genome, e.g., human coding regions, mammalian coding regions. For example, the probes may be a whole exome sequencing panel. In an embodiment, the probes may be a targeted panel of probes. In some cases, the panel may be a commercially available panel, such as whole exome or targeted sequencing panels available from Illumina.

After binding of the probes to coding sequencing still present in the DNA fragments, the probe-coding sequence complexes can be separated using probe separation techniques at step 110. For example, the probes may carry affinity tags (e.g., biotin), and the separation may be mediated by pulling the probe-coding sequence complexes out using affinity tag binders (e.g., avidin, streptavidin) linked to magnetic beads. This permits a separated of a probe-bound portion of the reaction mixture that includes the probe-coding sequence complexes from an unbound portion that includes coding sequence-depleted reannealed fragments that are not bound to probes. This unbound portion can be provided as Cot DA, e.g., Cot-1 DNA.

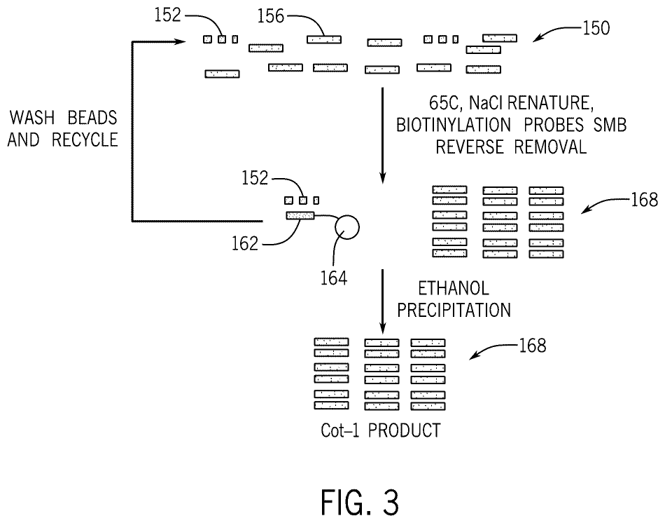

FIG. 3 is a schematic illustration of an embodiment of the process 100 showing fragment strands 150 (e.g., single strands of the fragments) generated from DNA fragments from a genomic DNA source. The strands 150 include a large portion of repetitive elements 156 and some presence of coding sequences 152. In an embodiment, the DNA fragments may be from a Cot DNA preparation. That is, the strands 150 may be generated from denaturing a Cot-1 DNA composition formed using conventional techniques, and the process 100 may be used to deplete coding sequences from prepared Cot-1 DNA to homogenize the composition and increase a fraction of repetitive elements 156 in the Cot-1 DNA after the process 100 is complete. In other embodiments, the strands 150 may be generated as part of an initial Cot-1 preparation technique using genomic DNA to generate fragments and after the fragmentation step is performed. The strands may be generated using denaturing temperatures as discussed herein.

The strands 150 are exposed to reannealing temperatures, e.g., 65° C., in the presence of an appropriate buffer to promote hybridization of probes 162 to any individual strands 150 including coding sequences 152 as well as reannealing of repetitive elements 156 in the strands 150 to one another based on complementarity. The coding sequences 152 that are bound to the probes 162 are not available for reannealing. The probes 162 may include probes to coding sequences 152 as well as their complements. However, in an embodiment, any unbound strands 150 may be digested using a single-stranded nuclease (e.g., S1, P1). The bound coding sequences 152 can be removed from the reannealed fragments 168 using probe affinity tags 164 coupled to beads (e.g., streptavidin magnetic beads, SMB), and the reannealed fragments 168 can be provided as the Cot-1 product 168. The reannealed fragments 168 may be precipitated as part of purification. Precipitation as discussed herein may refer to alcohol precipitation. In an embodiment, the ethanol precipitation is EtOH (0° C.)/3M NaOAC Precipitation, −20° C. overnight or −80° C. 1 hour.

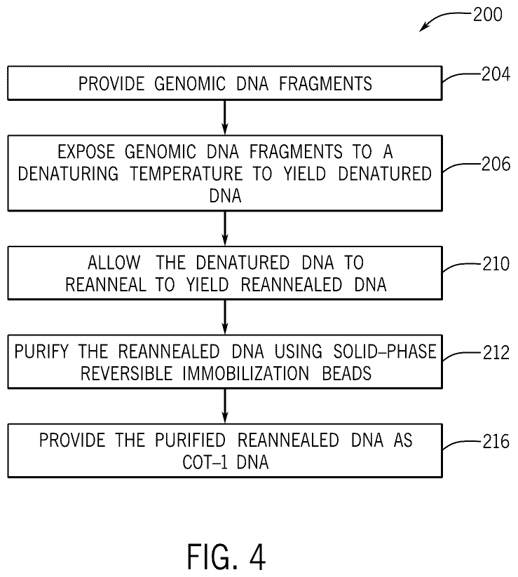

FIG. 4 is directed to a process 200 for improving Cot DNA quality. In embodiments, the process 200 may be used alone or in combination with other techniques discussed herein. For example, steps of the process 200 may be incorporated into the process 100 (FIG. 2). The process 200 may be used as part of a Cot-1 preparation to improve Cot-1 purity. The process 200 includes providing genomic DNA fragments at step 204. The fragments may be provided as generating discussed herein, via fragmentation of a genomic DNA source. The fragments are denatured at denaturing temperatures (e.g., above 90° C., 92° C.-100° C. for 10 minutes), and then allowed to reanneal under conditions that promote hybridization of repetitive elements (e.g., 60° C.-65° C., 60-120 minutes, reaction mixture concentration of 0.3-0.5M NaCl) at step 210. The reannealed DNA can be purified from single-stranded DNA in the reaction at step 212 using solid phase reversible immobilization beads (SPRI beads). SPRI beads function to preferentially bind to double-stranded or reannealed DNA relative to single-stranded DNA. Additionally, the single-stranded DNA may be digested using S1 or P1 nuclease that targets single-stranded nucleic acids. Both S1 and P1 nucleases cleave RNA and single stranded DNA with no base specificity. S1 nuclease is more active on DNA than RNA. Its reaction products are oligonucleotides or single nucleotides with 5′ phosphoryl groups. P1 nuclease cleaves its substrate at every position yielding nucleoside 5′ monophosphates, and it does not recognize or act on double-stranded DNA. Thus, using bead-based separation (e.g., magnetic pulldown and washing of supernatant), the purified reannealed DNA can be provided as Cot-1 DNA at step 216. For example, the reannealed DNA can be separated and precipitated. In an embodiment, the SPRI beads can be tuned to bind fragments of a particular size that corresponds to the genomic DNA fragments.

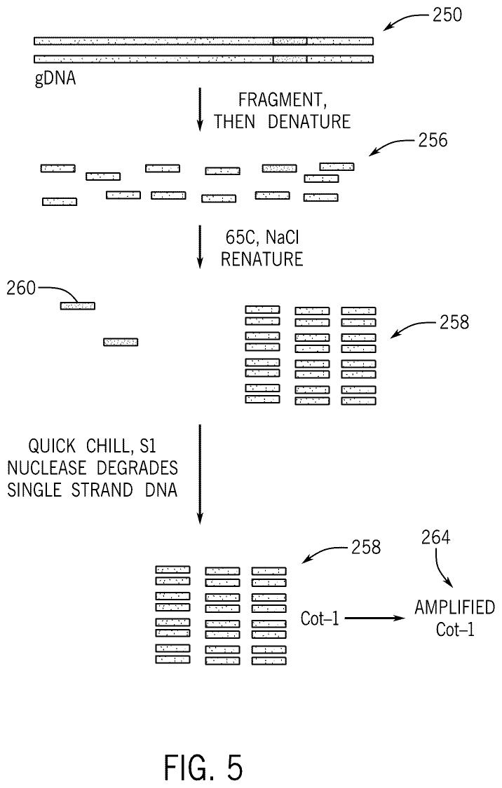

In another example, a Cot-1 preparation technique may include process steps that amplify desired fractions of Cot-1 to enrich the prepared Cot-1 for sequences or repetitive elements of interest. FIG. 5 is a schematic illustration of an embodiment of an enrichment workflow to generate Cot-1 DNA from genomic DNA 250. The genomic DNA 250 is fragmented and denatured to generate fragment strands 256 (e.g., single strands of the fragments). The strands 256 include a large portion of repetitive elements that reanneal to generate reannealed fragments 258 and with some non-annealed single fragment strands 260 remaining after the strands 256 are exposed to reannealing temperatures, e.g., 65° C., in the presence of an appropriate buffer to promote renaturing. The non-annealed single fragment strands 260 may be digested using a single-stranded nuclease (e.g., S1, P1). The reannealed fragments 258 can be amplified to generate amplified Cot-1 264 prior to precipitation. The amplification may be via random hexamer primers. In such an embodiment, the disparity between the repetitive element fraction and less abundant fractions in the reannealed fragments 258 can be exponentially increased such that the amplified Cot-1 264 is artificially enriched for the repetitive elements after amplification. In an embodiment the reannealed fragments 258 can be adapterized via library preparation techniques to attach specific adapters (e.g., Illumina sequencing adapters) via ligation and/or tagmentation as generally discussed in U.S. Pat. No. 11,788,139, which is hereby incorporated by reference herein. The adapters may include universal primer regions that can be used to amplify adapterized reannealed fragments 258 to generate the amplified Cot-1 264. In an embodiment the reannealed fragments 258 can be amplified using targeted primers designed in silico using known bioinformatic sequences for repetitive elements. The amplified Cot-1 264 may be processed and/or precipitated as generally discussed herein to remove components of the amplification reactions to generated purified amplified Cot-1 264. The amplified Cot-1 264 is a non-naturally occurring composition with a fractional distribution of repetitive elements that is modified or skewed outside of naturally occurring distributions.

A proposed function of Cot-1 in next generation sequencing (NGS) target enrichment assays is to prevent off-target capture of non-desirable repetitive elements. In some enrichment panels, probes with sequence identity to repetitive elements are unintentionally present. Cot-1 DNA, being composed entirely of repetitive elements, will hybridize to these unintentional probes and reduce or eliminate their ability to hybridize to and capture library fragments composed of repetitive sequence.

If enrichment probes targeting repetitive sequences are identified, there are various strategies to resolve their function. In one example, the probes having the unintentional repetitive element probe(s) can be removed from the panel. However, this is not applicable to panels that have already been established. Provided herein is a Cot-1 improvement technique in which common repetitive elements unintentionally probed in enrichment panels are identified bioinformatically. Based on this identification, sense and antisense oligos across these elements can be provided as a proxy for a synthetic Cot-1 and/or as an additive to Cot-1 as generally discussed herein. This approach can be applied to multiple panels that have already been physically manufactured that share common design strategies. This will result in a “minimal synthetic Cot-1” useful for target enrichment.

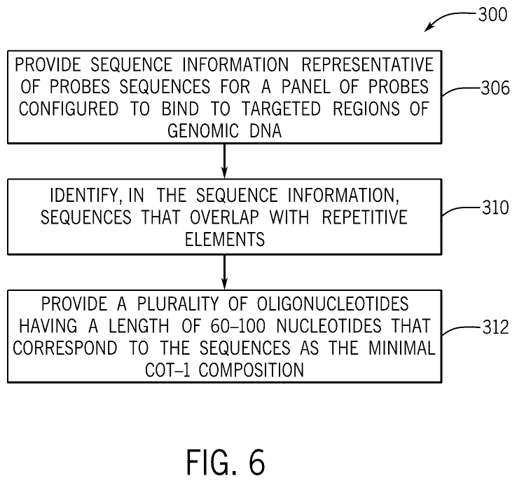

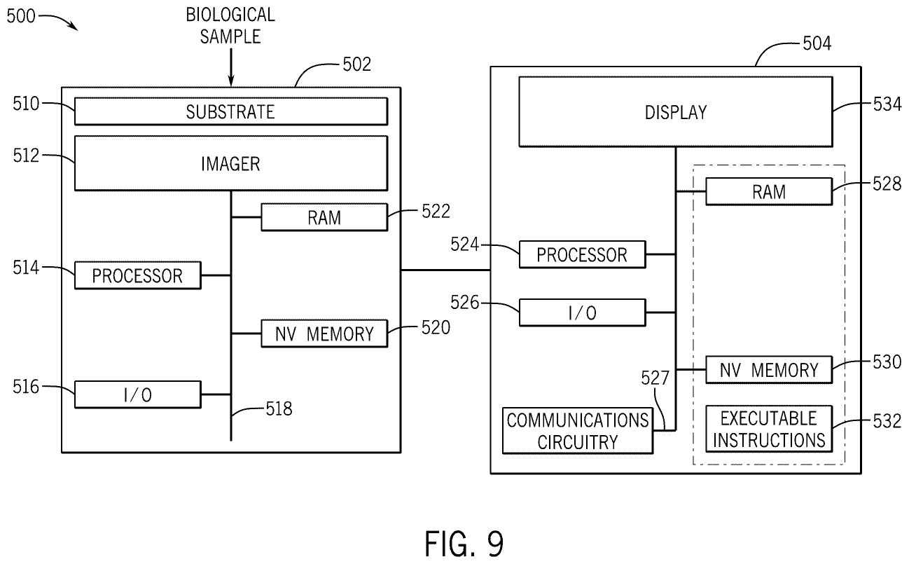

FIG. 6 is a flow diagram of a bioinformatic process 300 for designing and generating a minimal synthetic Cot-1. In an embodiment, the process including a step 306 of providing (e.g., accessing, transmitting, and/or receiving) information representative of probe sequences for a panel of probes of interest. For example, the probes may be configured to bind to targeted regions of a sample, which may be a genomic DNA sample or another nucleic acid sample source. The probes represent regions complementary to sequences in the sample DNA. In an embodiment, the probes may be 20 bp-120 bp in length. Using the sequencing information, sequences that overlap with repetitive elements in genomic DNA are identified at step 310. In an embodiment, the repetitive elements may be those of Table 1 or Table 2, by way of example. The genomic DNA may be a reference genome or a reference genome representation. In an embodiment, the overlap is identified using a database of characterized repetitive elements from a population. In an embodiment, the identification may be via a sequence analysis tool, such as a sequence analysis tool of a computer (see FIG. 9). The overlap may be based on one or more of sequence identity, sequence similarity, or sequence homology. In an embodiment, the sequences are scored based on homology, and sequences are designated as overlapping based on a homology score or value above a preset threshold. In an embodiment, the sequences are scored based on homology, and sequences are designated as overlapping based on a preset number of highest-homology sequences (e.g., highest 100 or 500).

After identification, the process 300 proceeds to a step 312 of providing a plurality of oligonucleotides having a length of 60-100 nucleotides that correspond to the sequences as the minimal Cot-1 composition. The corresponding sequences may include sense and antisense oligonucleotides. Providing the plurality of oligonucleotides may include synthesizing the oligonucleotides of the minimal synthetic Cot-1 composition. Providing the plurality of oligonucleotides may include transmitting sequence of the oligonucleotides of the minimal synthetic Cot-1 composition to a DNA synthesis facility and generating synthesis instructions to synthesize the oligonucleotides of the minimal synthetiCot-1 composition.

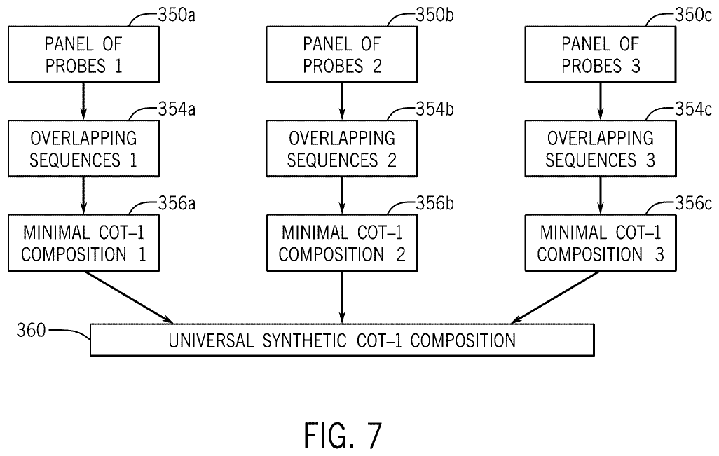

FIG. 7 is a schematic illustration of a universal synthetic Cot-1 composition that may be used as part of a reaction mixture for various probe panels (illustrated as probe panels 350a, 350b, 350c by way of example) that may have probes that target repetitive elements. For sequences of each of the probes of the various panels, the overlapping sequences 354a, 354b, 354c may be identified and used to design respective oligonucleotides of minimal Cot-1 compositions 356a, 356b, 356c. Pooling these oligonucleotides yields a universal synthetic Cot-1 composition 360. In an embodiment, the overlapping sequences 354a, 354b, 354c may be subject to additional processing and analysis by pooling the overlapping sequences 354a, 354b, 354c in silico and removing duplicates or generating a representative composition of the pool before progressing to synthesis of the universal synthetic Cot-1 composition 360.

The minimal synthetic Cot-1 composition and/or the universal synthetic Cot-1 composition as discussed herein may include single-stranded or double-stranded oligonucleotides. The minimal synthetic Cot-1 composition and/or the universal synthetic Cot-1 composition may be provided as a reagent or as part of a kit for targeted enrichment NGS techniques. In an embodiment, the minimal synthetic Cot-1 composition and/or the universal synthetic Cot-1 composition may be used in conjunction with conventional Cot-1 and/or the improved Cot-1 compositions as discussed herein.

### Examples

Sequences of a panel of interest (composed of 39,759 target enrichment probes) were evaluated with RepeatMasker (version 4.1.2-p1 Smit, A, R Hubley, and P Green. “RepeatMasker.” https://repeatmasker.org/.) using the NCBI/RMBLAST search engine (version 2.10.0+) using a curated database of repetitive elements (Dfam version 3.3 Storer, Jessica, Robert Hubley, Jeb Rosen, Travis J. Wheeler, and Arian F. Smit. “The Dfam Community Resource of Transposable Element Families, Sequence Models, and Genome Annotations.” Mobile DNA 12, no. 1 (Jan. 12, 2021): 2. https://doi.org/10.1186/s13100-020-00230-y). Resulting output from RepeatMasker was parsed to obtain the number of probes in the enrichment panel with homology to repetitive element classes ( ). Individual repeat elements within repeat classes were matched to the corresponding entries in Dfam. Only repeat elements with sequence identity to ≥30 probes from the panel of interest were considered ( ). In Table 2, Alpha/ALR elements were not targeted in panel of interest, but manually added as they are common repeat elements in the human genome.

The disclosed repetitive elements as discussed herein may be, for example, those referred to in Table 1 or Table 2. The disclosed minimal synthetic Cot-1 may include oligonucleotides in any combination of SEQ ID NOS: 1-1354.

TABLE 1
Repeat class and number of enrichment probes with
sequence homology within the panel of interest

| | Number of probes in “panel of interest” with |
|---|---|
| | significant sequence identity to indicated Repeat |
| Repeat Class | Class |
| LTR/ERVK | 1 |
| Satellite/centr | 1 |
| scRNA | 1 |
| SINE/5S-Deu-L2 | 1 |
| SINE/tRNA-Deu | 1 |
| snRNA | 1 |
| Unknown | 1 |
| DNA/TcMar-Tc1 | 2 |
| LTR | 2 |
| DNA/hAT | 3 |
| DNA/TcMar-Tc2 | 4 |
| SINE/tRNA | 4 |
| SINE/tRNA-RTE | 4 |
| DNA/hAT- | 5 |
| Blackjack | |
| Satellite | 5 |
| srpRNA | 6 |
| Retroposon/SVA | 9 |
| DNA/hAT-Tip100 | 10 |
| LINE/CR1 | 14 |
| LTR/ERVL | 43 |
| LTR/ERV1 | 49 |
| Low_complexity | 76 |
| LINE/L2 | 82 |
| DNA/TcMar-Tigger | 83 |
| LTR/ERVL-MaLR | 86 |
| DNA/hAT-Charlie | 114 |
| SINE/MIR | 144 |
| LINE/L1 | 485 |
| Simple_repeat | 683 |
| SINE/Alu | 948 |

TABLE 2
Repeat elements and number of enrichment
probes targeting panel of interest

| Repeat Element | | Representative sequence accessions |
|---|---|---|
| Name | Count | of repeat element in Dfam |
| AluSq | 30 | DF0000043.4 |
| L2 | 30 | DF0000359.4 |
| | | DF0000360.4 |
| | | DF0001161.2 |
| | | DF0001198.2 |
| | | DF0001250.2 |
| | | DF0001280.2 |
| | | DF0001333.2 |
| L1M4 | 32 | DF0000245.4 |
| | | DF0000246.4 |
| | | DF0000247.4 |
| | | DF0000248.4 |
| | | DF0000249.4 |
| | | DF0000250.4 |
| AluSz | 34 | DF0000051.4 |
| | | DF0000052.4 |
| AluSc | 36 | DF0000003.4 |
| | | DF0000037.4 |
| | | DF0000038.4 |
| FRAM | 36 | DF0000147.4 |
| AluSx | 37 | DF0000047.4 |
| | | DF0000048.4 |
| | | DF0000049.4 |
| | | DF0000050.4 |
| MIRb | 40 | DF0000975.4 |
| L1MC | 42 | DF0000010.4 |
| | | DF0000275.4 |
| | | DF0000276.4 |
| | | DF0000277.4 |
| | | DF0000278.4 |
| | | DF0000279.4 |
| | | DF0000280.4 |
| | | DF0000281.4 |
| | | DF0000282.4 |
| | | DF0000283.4 |
| | | DF0000284.4 |
| | | DF0000285.4 |
| L1M5 | 44 | DF0000008.4 |
| AluY | 46 | DF0000002.4 |
| | | DF0000053.4 |
| | | DF0000054.4 |
| | | DF0000055.4 |
| | | DF0000056.4 |
| | | DF0000057.4 |
| | | DF0000058.4 |
| | | DF0000060.4 |
| | | DF0000063.4 |
| | | DF0000064.4 |
| | | DF0000065.4 |
| | | DF0000066.4 |
| | | DF0000634.4 |
| | | DF0001145.2 |
| | | DF0001154.2 |
| | | DF0001169.2 |
| | | DF0001174.2 |
| | | DF0001197.2 |
| | | DF0001240.2 |
| | | DF0001316.2 |
| | | DF0001317.2 |
| | | DF0001318.2 |
| | | DF0001320.2 |
| | | DF0001321.2 |
| AluSg | 47 | DF0000039.4 |
| | | DF0000040.4 |
| | | DF0000041.4 |
| AluSc8 | 52 | DF0000038.4 |
| L1P3 | 52 | DF0000318.4 |
| | | DF0000319.4 |
| | | DF0000320.4 |
| AluJo | 54 | DF0000034.4 |
| MIR | 57 | DF0000001.4 |
| | | DF0000974.4 |
| | | DF0000975.4 |
| | | DF0000976.4 |
| AluSp | 72 | DF0000042.4 |
| Alu | 109 | DF0000002.4 |
| | | DF0000003.4 |
| | | DF0000007.4 |
| | | DF0000016.4 |
| | | DF0000034.4 |
| | | DF0000035.4 |
| | | DF0000036.4 |
| | | DF0000037.4 |
| | | DF0000038.4 |
| | | DF0000039.4 |
| | | DF0000040.4 |
| | | DF0000041.4 |
| | | DF0000042.4 |
| | | DF0000043.4 |
| | | DF0000044.4 |
| | | DF0000045.4 |
| | | DF0000046.4 |
| | | DF0000047.4 |
| | | DF0000048.4 |
| | | DF0000049.4 |
| | | DF0000050.4 |
| | | DF0000051.4 |
| | | DF0000052.4 |
| | | DF0000053.4 |
| | | DF0000054.4 |
| | | DF0000055.4 |
| | | DF0000056.4 |
| | | DF0000057.4 |
| | | DF0000058.4 |
| | | DF0000060.4 |
| | | DF0000063.4 |
| | | DF0000064.4 |
| | | DF0000065.4 |
| | | DF0000066.4 |
| | | DF0000073.4 |
| | | DF0000143.4 |
| | | DF0000144.4 |
| | | DF0000147.4 |
| | | DF0000634.4 |
| | | DF0001067.4 |
| | | DF0001068.4 |
| | | DF0001069.4 |
| | | DF0001070.4 |
| | | DF0001071.4 |
| | | DF0001072.4 |
| | | DF0001145.2 |
| | | DF0001154.2 |
| | | DF0001169.2 |
| | | DF0001174.2 |
| | | DF0001197.2 |
| | | DF0001240.2 |
| | | DF0001316.2 |
| | | DF0001317.2 |
| | | DF0001318.2 |
| | | DF0001320.2 |
| | | DF0001321.2 |
| AluJb | 150 | DF0000007.4 |
| Alpha/ALR | n/a | DF0000014.4 |
| | | DF0000015.4 |
| | | DF0000029.4 |

The identification of repetitive element sequences from Dfam and selection of k-mer oligonucleotides (80-mer oligonucleotides in this example) representing minimal synthetic Cot-1 were as follows. Sequences for each of the Dfam accessions (Table 2) were retrieved from the curated Dfam database version 3.6 (file: Dfam_curatedonly.embl.gz). Dfam sequences were divided into 80-mer, non-overlapping segments using a custom Python script. Resulting 80-mers were binned into clusters by sequence similarity and a best representative sequence was chosen using BBTools dedupe.sh with the following parameters: minoverlap=60, k=17, findoverlap=t, minidentity=80, cluster=t, pbr=t, absorbrc-t, pto=t

The resulting 682 representative sequences were processed to replace any observed ambiguous base (“N”, which represents either A, T, G, or C) were replaced with A (adenosine). The reverse-complement of the 682 sequences were added to the set, resulting in 1364 80-mer sequences (SEQ ID NOS: 1-1364, Table 3) representing both strands of DNA to comprise a minimal synthetic Cot-1 to test with the panel of interest. Oligonucleotides of (SEQ ID NOS: 1-1364) were synthesized as a concentrated pool for full-functional testing along with the panel of interest.

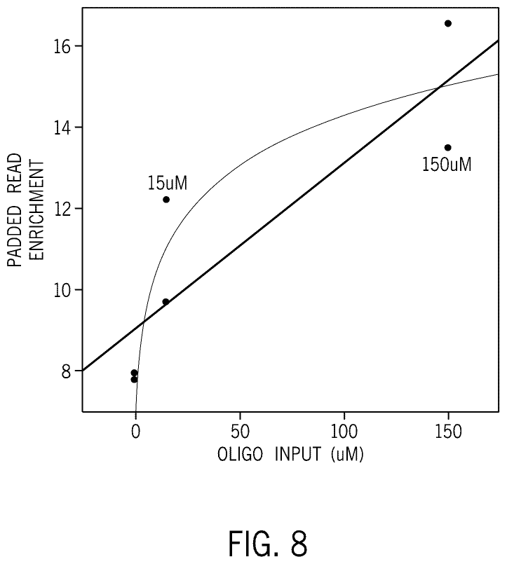

FIG. 8 shows the use of the minimal synthetic Cot-1 had a positive impact on padded read enrichment, a measure of on-target performance in NGS target enrichment assays. Padded read enrichment was measured using 0, 15, and 150 μM concentration of minimal synthetic Cot-1 oligonucleotides provided as a hybridization blocker in a target enrichment reaction using a probe panel of interest.

The techniques disclosed herein may be implemented in conjunction with a sequencing device and/or a sequence analysis device. For example, the steps of FIG. 6, including the identification of overlapping sequences, may be performed using a sequence analysis tool of a sequence analysis device. It should be understood that the disclosed sequence analysis may include analysis of thousands or millions of nucleotide bases and/or thousands or millions of sequence combinations. FIG. 9 is a schematic diagram of a sequencing device 500 that may be used in conjunction with the disclosed embodiments for acquiring sequencing data of overlapping as generally discussed herein. The sequence device 500 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. In an embodiment, the sequencing device 500 includes a separate sample processing device 502 and an associated computer 504. However, as noted, these may be implemented as a single device. Further, the associated computer 504 may be local to or networked or otherwise in communication with the sample processing device 502. In the depicted embodiment, the biological sample may be loaded into the sample processing device 502 on a sample substrate 510, e.g., a flow cell or slide, that is imaged to generate sequence data. For example, reagents that interact with the biological sample fluoresce at particular wavelengths in response to an excitation beam generated by an imager 512 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imager 512.

The imager detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imager 512 may be under processor control, e.g., via a processor 514, and the sample receiving device 502 may also include I/O controls 516, an internal bus 518, non-volatile memory 520, RAM 522 and any other memory structure such that the memory is capable of storing executable instructions, and other suitable hardware components that may be similar to those described with regard to FIG. 31. Further, the associated computer 504 may also include a processor 524, I/O controls 526, communications circuitry 527, and a memory architecture including RAM 528 and non-volatile memory 530, such that the memory architecture is capable of storing executable instructions 532. The hardware components may be linked by an internal bus, which may also link to the display 534. In embodiments in which the sequencing device 500 is implemented as an all-in-one device, certain redundant hardware elements may be eliminated.

The processor 514, 524 may be programmed to assign individual sequencing reads to a sample based on the associated index sequence or sequences according to the techniques provided herein. In particular embodiments, based on the image data acquired by the imager 512, the sequencing device 500 may be configured to generate sequencing data that includes base calls for each base of a sequencing read. Further, based on the image data, even for sequencing reads that are performed in series, the individual reads may be linked to the same location via the image data and, therefore, to the same template strand. In this manner, index sequencing reads may be associated with a sequencing read of an insert sequence before being assigned to a sample of origin. The processor 514, 524 may also be programmed to perform downstream analysis on the sequences corresponding to the inserts for a particular sample subsequent to assignment of sequencing reads to the sample.

Throughout this application various publications, patents and/or patent applications have been referenced. The disclosure of these publications in their entireties is hereby incorporated by reference in this application. The term comprising is intended herein to be open-ended, including not only the recited elements, but further encompassing any additional elements. While only certain features of the invention have been illustrated and described herein, many modifications and changes will occur to those skilled in the art. It is, therefore, to be understood that the appended claims are intended to cover all such modifications and changes as fall within the true spirit of the invention. Further, elements of the disclosed embodiments may be combined or exchanged. Accordingly, other embodiments are within the scope of the following claims.

1. A method of generating Cot-1 DNA, comprising:
contacting genomic DNA fragments with a hybridization probe panel under conditions that permit hybridization of probes of the hybridization probe panel to the genomic DNA fragments, wherein the probes of hybridization probe panel are configured to hybridize to coding regions of the genomic DNA fragments;
separating a first portion of the genomic DNA fragments from a second portion of the genomic DNA fragments based on differential binding to the hybridization probe panel, wherein the first portion is not bound to the probes and the second portion is bound to the probes; and
providing the first portion of the genomic DNA fragments as Cot-1 DNA.

2. The method of claim 1, comprising fragmenting genomic DNA by sonicating or shearing the genomic DNA to generate the genomic DNA fragments.

3. The method of claim 1, wherein the genomic DNA comprises human placental DNA.

4. The method of claim 1, wherein the conditions that permit hybridization comprise exposing the genomic DNA fragments to one or more cycles of denaturing temperatures and reannealing temperatures.

5. The method of claim 1, wherein the conditions that permit hybridization comprise exposing the genomic DNA fragments to a range of denaturing temperatures and subsequently holding a reannealing temperature.

6. The method of claim 1, comprising exposing the first portion to a denaturing temperature and a subsequent reannealing temperature before providing the first portion of the genomic DNA fragments as Cot-1 DNA.

7. The method of claim 1, comprising contacting the first portion with a single-stranded nuclease before providing the first portion of the genomic DNA fragments as Cot-1 DNA.

8. The method of claim 1, wherein separating the first portion from the second portion comprises using affinity tags coupled to the individual probes.

9. The method of claim 8, wherein separating the first portion from the second portion comprises using affinity tag binders coupled to magnetic beads, wherein the affinity tag binders are configured to bind to the affinity tags.

10. The method of claim 9, comprising washing the affinity tag binder coupled to the magnetic beads for reuse.

11. The method of claim 1, comprising precipitating the first portion before providing the first portion of the genomic DNA fragments as Cot-1 DNA.

12. The method of claim 1, wherein the Cot-1 DNA is at least 95% double-stranded DNA.

13. The method of claim 1, wherein the hybridization probe panel comprises an exome panel or a whole exome panel.

14. A Cot DNA composition prepared by a method comprising:
contacting genomic DNA fragments with a hybridization probe panel under conditions that permit hybridization of probes of the hybridization probe panel to the genomic DNA fragments, wherein the probes of hybridization probe panel are configured to hybridize to coding regions of the genomic DNA fragments;
separating a first portion of the genomic DNA fragments from a second portion of the genomic DNA fragments based on differential binding to the hybridization probe panel, wherein the first portion is not bound to the probes and the second portion is bound to the probes; and
providing the first portion of the genomic DNA fragments as the Cot DNA composition.
